(19) United States
(12) Patent Application Publication
(10) Pub. No.: US 2008/0188703 A1
(43) Pub. Date: Aug. 7, 2008

(54) Method for Producing a Stream of Hydrocarbons Containing from 5 to 12 Carbon Atoms Per Molecule and Having an Increased Content in Linear Alpha-Olefins

(75) Inventors: Thomas Hill, Ludwigshafen (DE); Christian Miller, Deidesheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/910,020

(22) PCT Filed: Mar. 28, 2006
(86) PCT No.: PCT/EP2006/061077

§ 371 Date: Sep. 28, 2007

(30) Foreign Application Priority Data
Mar. 29, 2005 (DE) 10 2005 014 177.3

(51) Int. Cl. C07C 5/22

(52) U.S. Cl. 585/671

(57) ABSTRACT

A process is proposed for obtaining a stream of aliphatic hydrocarbons comprising from 5 to 12 carbon atoms per molecule, with an increased proportion of linear α-olefins compared to a feed stream of aliphatic hydrocarbons comprising from 5 to 12 carbon atoms per molecule, with a content of linear α-olefins, linear internal olefins and dienes, wherein
the feed stream is fed to a first distillation zone D1 having at least 5 theoretical plates,
in which the linear α-olefins are removed partly or fully as a component of a vapor stream which additionally also comprises the dienes,
and at whose lower end a liquid stream is obtained which has been depleted partly or fully of linear α-olefins,
the liquid stream from the lower end of the first distillation zone D1 is introduced into a isomerization unit which is equipped with an isomerization catalyst over which the linear internal olefins are isomerized partly or fully to linear α-olefins, and the linear α-olefins formed in this way are removed as a component of a vapor stream ascending into the first distillation zone D1,
the vapor stream ascending out of the first distillation zone D1 enters a selective hydrogenation unit in which at least a portion of the dienes are hydrogenated selectively to olefins over a low-isomerization selective hydrogenation catalyst to obtain a vapor stream which is drawn off, condensed fully or partly and drawn off as a product stream with an increased proportion of linear α-olefins compared to the feed stream.

The invention relates to a process for obtaining a stream of hydrocarbons comprising from 5 to 12 carbon atoms per molecule, with an increased proportion of linear α-olefins compared to a feed stream of hydrocarbons comprising from 5 to 12 carbon atoms per molecule, with a content of linear α-olefins, linear internal olefins and dienes.

It is frequently desired to treat product streams from petrochemical processes, such as catalytic or thermal cracking, pyrolysis, oligomerization or Fischer-Tropsch syntheses, in such a way that the proportion of linear α-olefins is increased. For example, 1-butene is an important raw material for preparing copolymers, for example with ethylene, or for the synthesis of butene oxide.

Internal olefins, i.e. olefins having internal double bonds, may be isomerized over suitable isomerization catalysts to α-olefins, i.e. olefins having terminal double bonds. The equilibrium which is established is, however, strongly to the side of the internal olefins: for example, the thermodynamic equilibrium for the isomerization of 2-butenes to 1-butene is only about 14% 1-butene at a temperature of 200° C. and about 29% 1-butene at a temperature of 500° C. (cf. DE-A 103 11 139).

In order to increase the yield of α-olefins, the equilibrium position is therefore shifted by continuously removing the α-olefin product of value by distillation.

Such a reactive distillation process is described in U.S. Pat. No. 5,087,780: according to this, a hydrocarbon stream comprising 1-butene, 2-butenes and small proportions of butadiene is fed to a reactive distillation zone comprising a catalyst supported on alumina and having a palladium oxide active composition, and the butadiene is selectively hydrogenated with supply of hydrogen and the 2-butenes are isomerized to 1-butene. The catalyst may be used in the form of conventional distillation packings such as Raschig rings, Pall rings or in saddle form, or else in the form of palladium oxide on alumina extrudates, in pockets or as a loose bed in a column.

By distillatively withdrawing the lower-boiling 1-butene from the reaction zone, the isomerization equilibrium is shifted in the desired direction, i.e. in the direction toward the formation of further 1-butene product of value. A bottom stream richer in 2-butenes is drawn off and recycled partly back into the column in order to isomerize further fractions of 2-butenes to 1-butene.

The process has the particular disadvantage that the 1-butene already present in the feed stream is also introduced into the isomerization zone and is initially isomerized there to 2-butene to attain the thermodynamic equilibrium. In order to attain the desired enrichment of 1-butene in the top stream, however, the 2-butene obtained by unwanted isomerization has to be concentrated in an energetically demanding manner by high circulation through the bottom evaporator.

In the process of U.S. Pat. No. 6,768,038, a feed stream comprising at least one α-olefin and at least one olefin having an internal double bond is fed to an isomerization reaction zone comprising a catalyst bed, in which the α-olefins rise to the top of the column and the internal olefins are isomerized to α-olefins in contact with the fixed catalyst bed. In this case too, the isomerization equilibrium is shifted in the direction toward the α-olefin product of value by continuously removing it from the catalyst bed as a top stream of the column.

U.S. Pat. No. 6,242,662 describes a further process for preparing 1-butene from 2-butenes, in which a feed stream comprising at least one of the geometric isomers of 2-butene is distilled in a distillation zone which is connected to a hydroisomerization zone which is arranged at least partly outside the distillation zone. In one embodiment, the process also comprises a process step for removing butadienes which may be arranged before or after the distillation and hydroisomerization process steps connected to one another

It was accordingly an object of the invention to provide a more economically viable, especially more energetically favorable, process for obtaining a hydrocarbon stream with an increased proportion of α-olefins and decreased proportion of dienes compared to a feed stream.

The object is achieved by a process for obtaining a stream of hydrocarbons comprising from 5 to 12 carbon atoms per molecule, with an increased proportion of linear α-olefins and a lower proportion of dienes compared to a feed stream of aliphatic hydrocarbons comprising from 5 to 12 carbon atoms per molecule, with a content of linear α-olefins, linear internal olefins and dienes, wherein
the feed stream is fed to a first distillation zone D1 having at least 5 theoretical plates,
in which the linear α-olefins are removed partly or fully as a component of a vapor stream which additionally also comprises the dienes,
and at whose lower end a liquid stream is obtained which has been depleted partly or fully of linear α-olefins,
the liquid stream from the lower end of the first distillation zone D1 is introduced into a isomerization unit which is equipped with at least one isomerization catalyst over which the linear internal olefins are isomerized partly or fully to linear α-olefins, and the linear α-olefins formed in this way are removed as a component of a vapor stream ascending into the first distillation zone D1,
the vapor stream ascending out of the first distillation zone D1 enters a selective hydrogenation unit in which at least a portion of the dienes are hydrogenated selectively to olefins over a low-isomerization selective hydrogenation catalyst to obtain a vapor stream which is drawn off, condensed fully or partly and drawn off as a product stream with an increased proportion of linear α-olefins compared to the feed stream.

It has been found that it is possible to obtain a stream having an increased proportion of linear of α-olefins in a single process from a stream of hydrocarbons comprising linear α-olefins, linear internal olefins and dienes by distillatively removing the α-olefins from the feed stream, isomerizing the internal olefins to α-olefins and selectively hydrogenating the dienes to olefins.

The composition of the feed stream can vary within wide limits, but it comprises hydrocarbons having from 5 to 12 carbon atoms per molecule, preferably hydrocarbons having predominantly 5 carbon atoms per molecule or else having predominantly 6 carbon atoms per molecule. In this context, “predominantly” means that the feed stream comprises at least 90% by weight or at least 95% by weight or else at least 98% by weight of the corresponding hydrocarbons. In general, they are aliphatic hydrocarbons. C₅ hydrocarbon streams in particular may also comprise cycloaliphatic hydrocarbons.

The content of linear α-olefins is generally between 0.001 and 90% by weight, preferably between 10 and 70% by weight, more preferably between 30 and 60% by weight; the content of linear internal olefins is in the range from 5 to 99% by weight, preferably from 30 to 90% by weight, more preferably from 40 to 70% by weight, and the content of dienes is in the range from 0.001 to 5% by weight, preferably from 0.005 to 2% by weight.

The table specifies the typical components of a C₆ cut with the corresponding ranges from the proportions by weight, and also an exemplary composition in the last column:

| Component | | Range | Example |
|---|---|---|---|
| 2-methylpentene-1 | [%] | 0.001-10 | 0.21 |
| hexene-1 | [%] | 0.001-10 | <0.005 |
| n-hexane, | [%] | 0.001-40 | <0.001 |
| trans-3-hexene | [%] | 0.1-99.5 | 82.2 |
| cis-3-hexene | [%] | 0.1-50 | 14.5 |
| trans-2-hexene | [%] | 0.001-10 | 0.07 |
| 2-methylpentene-2 | [%] | 0.001-10 | 1.8 |
| cis-2-hexene | [%] | 0.001-10 | 0.02 |
| hexadiene | [%] | 0.001-5 | 0.003 |
| further C₆ components | [%] | 0.001-50 | 0.3 |
| C₅ components | [%] | 0.001-10 | 0.9 |
| C₇ components | [%] | 0.001-10 | 0.2 |

The hexadienes are present in the forms of different isomers. For example, the two conjugated double bond isomers 1,3- and 2,-4-hexadiene occur. Of the two isomers, it is possible in turn to detect the cis and the trans form. The nonconjugated 1,5-hexadiene may likewise be present.

The feed stream is fed to a first distillation zone D1 which is equipped with customary separating internals, in particular trays or structured packings. The distillation zone D1 is designed in such a way that the linear α-olefins are removed partly or fully as components of a vapor stream which additionally also comprises the dienes.

According to the invention, the first distillation zone is designed in such a way that it has at least 5 theoretical plates. The inventors have recognized that it is necessary for the effective, especially energetically advantageous, enrichment of linear α-olefins to remove them from the feed stream in a distillation zone which has sufficient separating action for the very substantial removal of linear α-terminal olefins from linear internal olefins, especially because the volatilities of linear-α-terminal and internal olefins differ only slightly.

The first distillation zone D1 preferably comprises at least 10 theoretical plates, more preferably from 20 to 60 theoretical plates, in particular 40 theoretical plates.

At the lower end of the first distillation zone, a liquid stream is obtained which has been partly or fully depleted of linear α-olefins. This liquid stream is introduced into an isomerization unit which is equipped with an isomerization catalyst over which the linear internal olefins are isomerized partly or fully to linear α-olefins, and the linear α-olefins formed in this way are removed as a component of a vapor stream ascending into the first distillation zone D1.

The isomerization unit preferably comprises a first reactive distillation zone RD1 which is integrated in the reactive distillation column RDK. In the first reactive distillation zone RD1, a hydroisomerization catalyst or an acidic or basic isomerization catalyst may preferably be used. For this purpose, all catalysts known from the prior art may be used.

Alternatively or additionally to the first reactive distillation zone RD1, the isomerization unit may also comprise an intermediate reactor ZR1, a liquid stream from below the first distillation zone D1 in the reactive distillation column RDK being passed partly or fully into the intermediate reactor ZR1 and an isomerization of the olefins is carried out in said intermediate reactor, if appropriate with supply of hydrogen.

In a first variant, the isomerization catalyst is a weakly acidic to weakly basic catalyst, especially based on zeolite or based on alumina.

Suitable for this purpose are in particular alkaline earth metal oxides on alumina, as described in EP-A 718 036, mixed alumina-silica supports which are doped with oxides of the alkaline earth metals, boron group metals, lanthanides or elements of the iron group (U.S. Pat. No. 4,814,542), or γ-alumina plated with alkali metals which is described in JP 51-108691. Also suitable are catalysts composed of manganese oxide on alumina, described in U.S. Pat. No. 4,289,919, catalysts composed of magnesium oxides, alkali metal oxides or zircon oxides dispersed on an alumina support, described in EP-A 234 498, and alumina catalysts which additionally comprise sodium oxide and silicon oxide, described in U.S. Pat. No. 4,229,610.

Suitable catalysts based on zeolite are in particular boro- or aluminosilicates whose acidity has been reduced by means of ion exchange (exchange of hydrogen for alkali metals, alkaline earth metals or transition metals). Such catalysts are described, for example, in U.S. Pat. No. 3,475,511 or DE 129 900. Suitable zeolite-based catalysts are also described in EP-A 1 298 99 (zeolites of the pentasil type). Also suitable are molecular sieves exchanged with alkali metals or alkaline earth metals (described in U.S. Pat. No. 3,475,511), alumosilicates (described in U.S. Pat. No. 4,749,819), and also zeolites in alkali metal or alkaline earth metal form (described in U.S. Pat. No. 4,992,613) and those based on crystalline borosilicates (described in U.S. Pat. No. 4,499,326).

On the subject of zeolite-based catalysts, the following data relating to conversion, selectivity and space time yield can be taken from DE 129 900;

Catalyst: sodium- or zinc-doped ZSMS or ZBM11/10,

Hourly space velocity: from 1 to 5 kg of 2-butene/h at from 250 to 450° C., space-time yield based on 1-butene from 0.2 to 1.5 kg/h, depending on hourly space velocity, 1-butene selectivity from 98 to 99% and lifetime >10 days without noticeable deactivation, but longer lifetimes were not tested.

In addition, catalysts based on alumina can be used, predominantly based on γ-alumina which has been doped with alkali metals, alkaline earth metals or transition metals. Such catalysts are described, for example, in EP 751 106 or EP 718 036. With regard to conversion, selectivity and space-time yield, the following data can be taken from EP 718 036:

Catalysts: 6% strontium oxide on γ-alumina, hourly space velocity: 5 kg of 2-butene/h at from 450 to 500° C., space-time yield based on 1-butene from 1 to 1.5 kg/h depending on the temperature, 1-butene selectivity >99%.

Furthermore, it is also possible to use other known isomerization catalysts, for example phosphate-based heterogeneous catalysts, nickel sulfide, nickel oxide or zinc/iron chromate catalysts.

The above-described isomerization catalysts based on zeolite or based on alumina, or the above-described phosphate-based heterogeneous catalysts, nickel sulfide, nickel oxide or zinc/iron chromate catalysts, are used preferably at elevated temperature, in particular in the range between 250 and 500° C., especially in the intermediate reactor ZR1, since the equilibrium position with regard to 1-butene is more advantageous at elevated temperature.

In a preferred variant, the isomerization catalysts are used hydroisomerization catalysts, i.e. supported catalysts, frequently on alumina supports, with a palladium active composition and, if appropriate, one or more dopants. The undesired hydrogenation activity of the isomerization catalyst can be attenuated by adding one or more additives, especially sulfur or a sulfur compound. Since the catalytically active species in the hydroisomerization is formed with hydrogen, it is in principle always necessary when using hydroisomerization catalysts to feed a hydrogen stream, generally of from about 0.1 to 1 mol % based on the total content of butenes. Hydroisomerization catalysts are described, for example, in EP 930 285, U.S. Pat. No. 5,087,780 or U.S. Pat. No. 6,156,947.

Suitable hydroisomerization catalysts in the intermediate reactor ZR1 are conventional supported catalysts, especially on a support composed of alumina, silicon dioxide, titanium dioxide, zirconium dioxide, silicon dioxide/alumina, calcium carbonate, silicon carbide, activated carbon and combinations thereof. It is possible to use all common, industrially producible catalyst forms, especially extrudates, spheres, tablets, annular tablets, hollow extrudates or trilobes. The dimensions of the catalyst moldings vary in relation to their diameter especially between 1 and 5 mm, more preferably between 2 and 4 mm. The catalyst moldings are saturated, dried and calcined preferably as described in EP-A 0 992 284.

After the calcination, the catalyst is in principle ready for use, but can, if required or desired, before use for selective hydrogenation, be activated by prereduction in a known manner and, if appropriate, also again be surface-passivated.

The metal content on the catalyst is typically between 0.01 and 2.0% by weight of palladium based on the total weight of the catalyst molding, preferably between 0.05 and 1.0% by weight, more preferably between 0.1 and 0.5% by weight.

The bulk density of the finished catalyst is generally between 500 and 1000 g/l.

The hydrogenation activity of the catalyst may be attenuated by adding an additive, in which case the additive can in particular be sulfur or a sulfur compound, selenium or a selenium compound, tellurium or a tellurium compound, as described in EP-A 841 090.

Useful hydroisomerization catalysts for use in the first reactive distillation zone RD1 in the reactive distillation column RDK are likewise in principle the conventional supported catalysts known from the prior art, to which the above specifications for the hydroisomerization catalysts usable in the intermediate reactor ZR1 apply in principle, but particular preference is given to lower diameters of the catalyst moldings, in particular between 0.5 and 5 mm, more preferably between 1 and 3.5 mm.

Hydroisomerization catalysts may particularly advantageously be used in the form of thin-layer catalysts, as described, for example, in EP-A 827 944. These have excellent distillative and catalytic action. They are catalyst packings which can be prepared by applying at least one substance active as a catalyst and/or promoter to woven fabric or foils as a support material.

The support material used for the thin-layer catalysts may be a multitude of foils and woven fabrics, and also knitted fabrics. It is possible in accordance with the invention to use woven fabrics with different weave type, such as plain weave, denim Dutch weave, five-shaft satin weave or else other specialty weaves. In one embodiment of the invention, useful woven meshes are woven from weavable metal wires such as iron, spring steel, brass, phosphor bronze, pure nickel Monel, aluminum, silver, nickel silver, nickel, chromium nickel, chromium steel, nonrusting, acid-resistant and high-temperature-resistant chromium nickel steels, and also titanium.

It is likewise possible to use woven fabrics made of inorganic materials, such as Al₂O₃ and/or SiO₂.

It is also possible in one embodiment of the invention to use synthetic wires and woven fabrics made of polymers. Examples are polyamides, polyesters, polyvinyls, polyolefins such as polyethylene, polypropene, polytetrafluorethylene and other polymers which can be processed to woven fabrics.

Preferred support materials are metal foils or woven metal fabrics, for example stainless steels having the materials numbers 1.4767, 1.4401, 1.4610, 1.4765, 1.4847, 1.4301, etc. The designation of these materials with the material numbers specified follows the specifications of the materials numbers in the “stahleisenliste” [list of irons and steels], published by the Verein Deutscher Eisenhüttenleute, 8th edition, pages 87, 89 and 106, Verlag Stahleisen mBH, Dusseldorf, 1990. The material of material numbers 1.4767 is also known under the name Kanthal.

The metal foils and woven metal fabrics are particularly suitable since they can be roughened by a heat treatment on the surface before the coating with catalytically active compounds or promoters. To this end, the metallic supports are heated in oxyenous atmosphere such as air at temperatures of from 400 to 1100° C., preferably from 600 to 1000° C., for from 0.5 to 24 hours, preferably from 1 to 10 hours. In one embodiment of the invention, this pretreatment allows the activity of the catalyst to be controlled or increased.

The catalyst supports can be coated with catalytically active compounds and promoters by means of various processes.

In one embodiment, the substance is active as a catalyst and/or promoter are applied by impregnating the support with a solution comprising the substance or a precursor thereof, by electrochemical deposition or deposition in the presence of a reducing agent (electrolose deposition).

The woven catalyst fabric or the catalyst foil can then be shaped to monoliths.

The catalyst supports can be coated by means of vacuum deposition technology with “thin films” of catalytically active compounds and promoters. “Thin layers” refer to platings in the thickness range between a few Å (10⁻¹⁰ m) and a maximum of 0.5 μm. The vacuum deposition techniques employed in accordance with the invention may be various processes. Examples are thermal evaporation, flash evaporation, cathode atomization (sputtering) and the combination of thermal evaporation and cathode atomization. The thermal evaporation may be effected by direct or indirect electrical heating.

Evaporation by means of electron beam may likewise be used. To this end, the substance to be evaporated in a water-cooled crucible is surface-heated so strongly with an electron beam that even high-melting metals and dielectrics are evaporated.

The palladium content of the thin-layer catalysts is preferably between 0.01 and 1 g/l of packing volume, more preferably between 0.03 and 0.5 g/l of packing volume, in particular between 0.05 and 0.2 μg/l of packing volume.

The hydrogenation activity of the palladium thin-layer catalyst may be attenuated by adding an additive, especially by adding sulfur or a sulfur compound, selenium or a selenium compound, tellurium or a tellurium compound, or combinations thereof.

In the process variant in which the first reactive distillation zone RD1 is equipped with a hydroisomerization catalyst, a hydrogen stream has to be introduced into the reactive distillation column below the first reactive distillation zone RD1.

The vapor stream ascending out of the first distillation zone D1 enters a selective hydrogenation unit which preferably comprises a second reactive distillation zone RD2 in which at least a portion of the dienes are selectively hydrogenated to olefins over a low-isomerization selective hydrogenation catalyst.

The low-isomerization selective hydrogenation catalyst may in particular be a supported catalyst having an active composition based on palladium which is doped with one or more elements from group 1B, preferably with silver.

Within the reactive distillation column RDK, the low-isomerization selective hydrogenation catalyst is arranged in the second reactive distillation zone RD2 and may additionally be arranged in the prereactor and/or in the intermediate reactor ZR2. It may be identical to a distillation structure, or be configured as a thin-layer catalyst, or a woven fabric or knitted fabric coated and shaped to a distillation packing.

The process variant in which the first distillation zone D1, the first reactive distillation zone RD1 and the second reactive distillation zone RD2 are integrated in a single reactive distillation column RDK is particularly favorable energetically and also with regard to the capital costs.

Advantageously, the vapor stream ascending out of the second reactive distillation zone RD2 can be concentrated in linear α-olefins in a second distillation zone D2 which is likewise integrated in the reactive distillation column RDK.

The vapor stream ascending out of the second reactive distillation zone RD2 is, if appropriate after concentration in the second distillation zone D2, drawn off as the top stream of the reactive distillation column RDK, condensed in a condenser at the top of the column, preferably introduced partly as reflux back to the column and otherwise drawn off as a product stream having an increased proportion of linear α-olefins compared to the feed stream.

In a further process variant, a liquid stream is withdrawn from the reactive distillation column RDK above the first distillation zone D1 and introduced into an intermediate reactor ZR2 in which, with supply of hydrogen, a selective hydrogenation of the dienes to olefins is carried out over an appropriate catalyst, if appropriate with supply of further reactants. The further reactants may also be a substream of the feed stream to the reactive distillation column or another stream of hydrocarbons having the same number of carbon atoms as the feed stream. From the intermediate reactor ZR2, a liquid product stream is drawn off and can be recycled fully or partly into the reactive distillation column RDK above the first distillation zone D1.

For the selective hydrogenation of the dienes in the second reactive distillation zone RD2, it is necessary to feed hydrogen into the reactive distillation column; this has to be below the second reactive distillation zone RD2, and the precise location of the feeding can be different, for example above and/or below the first reactive distillation zone RD1.

In a further variant, the feed stream comprising hydrocarbons having from 5 to 12 carbon atoms per molecule, before the feeding to the first distillation zone D1 in the reactive distillation column RDK, may be conducted partly or fully through a prereactor VR in which an isomerization of the olefins or, with supply of hydrogen, a selective hydrogenation of the dienes is carried out. Additional catalyst and/or reactants may be metered into the prereactor. The prereactor may in particular be run under operating conditions which differ from those in the reactive distillation column RDK.

In a further advantageous variant, a liquid stream from the reactive distillation zone RD1 of the reactive distillation column RDK may be drawn off and introduced into an intermediate reactor ZR1 in which, if appropriate with supply of hydrogen, an isomerization of the olefins is carried out. The product stream obtained in this way is, likewise in liquid form, recycled into the reactive distillation column RDK, preferably into the first reactive distillation zone RD1. The process conditions and also the type of catalyst used in the intermediate reactor ZR1 in this case may differ compared to the first reactive distillation zone RD1 in the reactive distillation column RDK.

The process variants using intermediate reactors (ZR1 and/or ZR2) which are disposed outside the reactive distillation column RDK have the advantages that an increase in the residence time, other reaction conditions, for example with regard to pressure, temperature and catalysts compared to the reactive distillation column RDK, and also easier installation and deinstallation of the catalyst is possible. The intermediate reactors ZR1 and/or ZR2 may be heated and/or stirred.

The isomerization catalyst in the first reactive distillation zone RD1 and/or the low-isomerization selective hydrogenation catalyst in the second reactive distillation zone RD2 may each independently be present in the form of a coating of a distillation packing or in the form of catalyst particles which are introduced on a distillation tray and/or downcomer of a distillation tray or in a packing which is not catalytically active as such.

The type of this packing is not restricted, provided that it is designed in such a way that it can accommodate catalyst particles. Suitable examples are packings with pockets made of wire as distillation internals, such as the KATAPAK-S design from Sulzer A G, CH-8404 Winterthur, or which are designed as flat pockets which are inserted between the individual layers of the distillation packings, such as the MULTIPAK design from Montz GmbH, D-40723 Hilden. It is also possible to use so-called bales from CDTech, Houston, USA, which are described, for example, in EP-A 0 466 954.

When catalysts are to be used in the form of particles, it is particularly advantageous to use packings with interstices with first and second packing subregions which are arranged in an alternating manner and differ by their specific surface area, in such a way that the quotient of the hydraulic diameter for the gas stream through the packing and the equivalent diameter of the catalyst particles in the first subregions is in the range from 2 to 20, preferably in the range from 5 to 10, so that the catalyst particles are introduced into the interstices loosely under reaction of gravity, distributed and discharged, and the quotient of the hydraulic diameter for the gas stream through the packing and the equivalent diameter of the catalyst particles in the second packing subregions is <1, so that no catalyst particles are introduced into the second packing subregions. Such packings are described in WO 03/047747.

A third distillation zone for the purpose of enriching high boilers in the bottom of the distillation column RDK may preferably be arranged in the reactive distillation column RDK below the first reactive distillation zone RD1.

The energy is introduced into the reactive distillation column RDK advantageously via a bottom evaporator SV, or else additionally via external heat exchangers and/or by means of heat exchangers integrated into the separating internals of one or more of the distillation zones.

The reactive distillation column RDK preferably has between 10 and 200 theoretical plates, in particular between 30 and 120 theoretical plates.

The pressure at the top of the reactive distillation column RDK is preferably adjusted in such a way that the temperature in the column bottom is between 0 and 400° C., in particular between 50 and 10° C. Depending on the reaction pressure selected, this can be done with a vacuum pump and/or a pressure regulating device.

In the distillation zones D1, D2 and/or D3, separating internals having a high number of plates may be used advantageously, especially metal fabric packings or sheet metal packings with molded structure, for example of the types Sulzer Melapack®, Sulzer BX®, Montz B1® or Montz A3®, or else random packings or trays.

The process according to the invention has advantages especially with regard to capital costs and the energy input required.

The linear α-olefins from the feed stream are removed partly or fully before contact with the isomerization unit in the first distillation zone D1, as a result of which losses of α-olefins from the feed stream by isomerization to internal olefins are prevented. In addition, the dienes are also removed from the feed stream partly or fully in the first distillation unit D1 and thus do not come into contact with the isomerization catalyst Accordingly, damage thereto as a result of polymerization of the dienes over the acidic or basic sites with the consequence of fouling and deactivation of the catalyst are prevented.

The dienes are depleted in the first distillation unit D1 preferably down to from <5 to <1 000 ppm, more preferably down to from <5 to <250 ppm, more preferably down to from <5 to <100 ppm.

The invention is illustrated in detail below with reference to a drawing and to a working example.

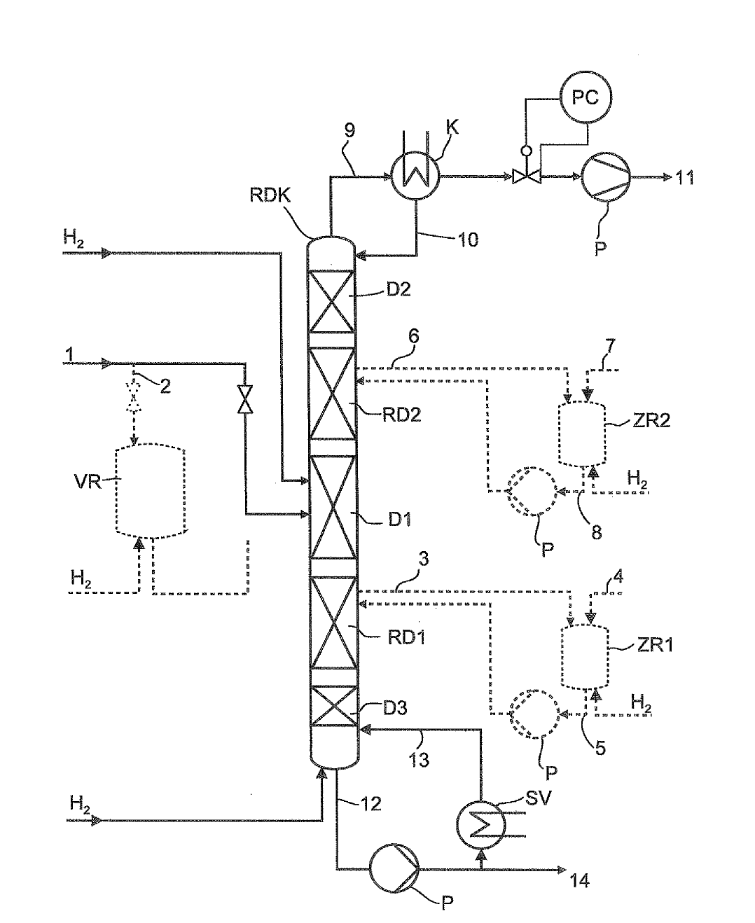

The sole FIG. 1 shows the schematic illustration of a preferred plant for carrying out the process according to the invention.

In this figure, plant parts which may optionally be provided are shown with broken lines.

A feed stream of hydrocarbons having from 5 to 12 carbon atoms per molecule, stream 1, is fed to the distillation zone D1 a reactive distillation column RDK.

Optionally, in one variant, a substream 2 of the feed stream 1 may be passed via a prereactor VR, in which, with supply of hydrogen which, as shown in the figure, can preferably be supplied in the lower region of the prereactor, a selective hydrogenation of the dienes and/or an isomerization of the olefins is carried out. Below the first distillation zone D1, a first reactive distillation zone RD1, in which an isomerization of the olefins is carried out, is advantageously arranged in the reactive distillation column RDK. In one variant which is shown in the figure with broken lines, a liquid stream or substream 3 is drawn off from the first reactive distillation zone RD1 and introduced into an intermediate reactor ZR1, into which a heterogeneous or homogeneous catalyst has been introduced and into which a stream 4 comprising catalyst and/or reactants can additionally be introduced.

When a hydroisomerization is carried out in the first intermediate reactor ZR1, hydrogen, H₂, additionally has to be introduced, for example, as in the preferred embodiment shown in the figure, via the lower region of the first intermediate reactor ZR1. From the first intermediate reactor ZR1, a liquid stream 5 is drawn off and is recycled by means of a pump P into the first reactive distillation zone RD1 of the reactive distillation column RDK.

Above the first distillation zone D1, a second reactive distillation zone RD2 is arranged in the reactive distillation column RDK, in which, in the presence of a low-isomerization selective hydrogenation catalyst and with supply of hydrogen, H₂, a selective hydrogenation of the dienes takes place in a region below the second reactive distillation zone RD2 as shown in the figure, for example in the first distillation zone D1 and in the lower region of the reactive distillation column RDK.

From the second reactive distillation zone D2, a liquid stream 6 can be drawn off and introduced into a second intermediate reactor ZR2 in which, with supply of hydrogen, in the lower region of the second intermediate reactor ZR2 in the preferred embodiment shown in the figure, a selective hydrogenation of the dienes takes place. If appropriate, a stream 7 comprising catalyst and/or reactants can be fed into the second intermediate reactor ZR2. From the second intermediate reactor ZR2, a liquid stream 8 is drawn off and recycled by means of a pump P into the second reactive distillation zone RD2.

In the preferred embodiment shown in the figure, above the second reactive distillation zone RD2, a second distillation zone D2 in which the α-olefins are concentrated further is arranged in the reactive distillation column RDK.

From the upper region of the reactive distillation column RDK, a top stream 9 which is enriched in α-olefins is drawn off, passed through a condenser K at the top of the column, introduced partly as reflux 10 back to the reactive distillation column RDK and otherwise drawn off as product stream 11.

In the lower region of the reactive distillation column RDK, a third distillation zone D3 is provided, in which enrichment of the high boilers takes place.

From the reactive distillation column RDK, a bottom stream 12 is drawn off, conveyed by means of a pump 3, recycled partly by means of a bottom evaporator SV as stream 13 back into the bottom region of the reactive distillation column RDK and otherwise discharged from the process as stream 14.

1-22. (canceled)

23. A process for obtaining a stream of hydrocarbons comprising from 5 to 12 carbon atoms per molecule, with an increased proportion of linear α-olefins and a lower proportion of dienes compared to a feed stream of hydrocarbons comprising from 5 to 12 carbon atoms per molecule, with a content of linear α-olefins, linear internal olefins and dienes, with supply of hydrogen, wherein
(a) the feed stream is fed to a first distillation zone D1 having at least 5 theoretical plates,
(b) in which the linear α-olefins are removed partly or filly as a component of a vapor stream which additionally also comprises the dienes,
(c) and at whose lower end a liquid stream is obtained which has been depleted partly or fully of linear α-olefins,
(d) the liquid stream from the lower end of the first distillation zone D1 is introduced into a isomerization unit which is equipped with at least one isomerization catalyst over which the linear internal olefins are isomerized partly or fully to linear α-olefins, and the linear α-olefins formed in this way are removed as a component of a vapor stream ascending into the first distillation zone D1,
(e) the vapor stream ascending out of the first distillation zone D1 enters a selective hydrogenation unit in which at least a portion of the dienes are hydrogenated selectively to olefins over a low-isomerization selective hydrogenation catalyst to obtain a vapor stream which is drawn off, condensed fully or partly and drawn off as a product stream with an increased proportion of linear α-olefins compared to the feed stream.

24. The process of claim 23, wherein the selective hydrogenation unit comprises a reactive distillation zone RD, and the distillation zone D1 and the reactive distillation zone RD2 are integrated in a single reactive distillation column RDK.

25. The process of claim 23, wherein the first distillation zone D1 comprises at least 10, preferably from 20 to 60, theoretical plates.

26. The process of claim 23, wherein the isomerization unit comprises a first reactive distillation zone RD1 which is integrated in the reactive distillation column RDK.

27. The process of claim 23, wherein the isomerization unit is formed from the first reactive distillation zone RD1, and the selective hydrogenation unit from the second reactive distillation zone RD2.

28. The process of claim 23, wherein the isomerization unit comprises an intermediate reactor ZR1, a liquid stream below the first distillation zone D1 being passed partly or fully into the intermediate reactor ZR1, and an isomerization of the olefins is carried out in said intermediate reactor.

29. The process of claim 23, wherein the selective hydrogenation unit comprises an intermediate reactor ZR2 into which a liquid stream is introduced and is drawn off above the distillation zone D1, and at least a portion of the dienes is selectively hydrogenated with supply of hydrogen in the intermediate reactor ZR2 to obtain a liquid stream which is drawn off as a product stream with an increased proportion of linear α-olefins compared to the feed stream, or is recycled fully or partly into the reactive distillation column RDK.

30. The process of claim 23, wherein a second distillation zone D2 is disposed in the upper region of the reactive distillation column RDK, and a further enrichment of linear α-olefins is effected in said second distillation zone to obtain, from the second distillation zone D2, a vapor stream which is condensed and drawn off as a product stream having an increased proportion of linear α-olefins compared to the feed stream.

31. The process of claim 24, wherein hydrogen is supplied into the reactive distillation column RDK below the second reactive distillation zone RD2.

32. The process of claim 31, wherein hydrogen is supplied below the first reactive distillation zone RD1 and/or above the first reactive distillation zone RD1.

33. The process of claim 23, wherein the isomerization unit is equipped with a hydroisomerization catalyst and the selective hydrogenation unit is equipped with a low-hydroisomerization selective hydrogenation catalyst.

34. The process of claim 23, wherein a third distillation zone D3 for removing high boilers via the bottom of the reactive distillation column RDK is provided below the first reactive distillation zone RD1 in the reactive distillation column RDK.

35. The process of claim 23, wherein the feed stream of hydrocarbons comprising from 5 to 12 carbon atoms per molecule, before it is fed to the reactive distillation column RDK, is passed partly or fully through a prereactor VR in which an isomerization of the olefins or, with supply of hydrogen, a selective hydrogenation of at least a portion of the dienes is carried out.

36. The process of claim 23, wherein the isomerization catalyst in the first reactive distillation zone RD1 and/or the low-isomerization selective hydrogenation catalyst in the second reactive distillation zone RD2 is present as a coating of a distillation packing or in the form of catalyst particles which are introduced on a distillation tray and/or in the downcomer of a distillation tray or in a packing.

37. The process of claim 23, wherein the separating internals in the first distillation zone D1 and, if appropriate, in the second distillation zone D2 are structured packings.

38. The process of claim 23, wherein the heat is supplied to the reactive distillation column RDK via a bottom evaporator SV.

39. The process of to claim 38, wherein the heat is supplied additionally via external heat exchangers and/or heat exchangers integrated into the separating internals.

40. The process of claim 23, wherein the catalyst introduced in the first reactive distillation zone RD1 is a supported catalyst with an active composition based on palladium.

41. The process of claim 40, wherein the hydrogenation activity of the catalyst in the first reactive distillation zone RD1 is attenuated by adding at least one additive.

42. The process of claim 23, wherein the catalyst in the second reactive distillation zone RD2 is a supported catalyst with an active composition based on palladium which has been doped with one or more elements from group 1b.

43. The process of claim 23, wherein the feed stream to the reactive distillation column RDK comprises hydrocarbons having predominantly 6 carbon atoms per molecule.

44. The process of claim 23, wherein the feed stream to the reactive distillation column RDK comprises hydrocarbons having predominantly 5 carbon atoms per molecule.
